(19) United States
(12) Patent Application Publication
(10) Pub. No.: US 2015/0306259 A1
(43) Pub. Date: Oct. 29, 2015

(54) PACKAGING UNIT FOR A PHARMACEUTICAL, MEDICAL, OR COSMETIC ITEM AND METHOD OF STERILIZING A PHARMACEUTICAL, MEDICAL, OR COSMETIC ITEM THAT CAN BE ARRANGED IN THE PACKAGING UNIT

(71) Applicant: SCHOTT AG, Mainz (DE)

(72) Inventors: Gregor Deutschle, Wiesbaden (DE); Edgar Pawlowski, Stadecken-Elsheim (DE); Joern Wassenberg, Mainz (DE); Isabell Dubrau, St. Gallen (CH); Judith Auerbach, Niederteufen (CH)

(21) Appl. No.: 14/696,078

(22) Filed: Apr. 24, 2015

(30) Foreign Application Priority Data
Apr. 24, 2014 (DE) 10 2014 105 787.2

(51) Int. Cl. A61L 2/00; B65D 65/38; B65D 75/26; A61B 19/02; A61L 2/20; B65D 33/25

(52) U.S. Cl. CPC A61L 2/0094; A61L 2/206; A61L 2/204; B65D 33/2508; B65D 33/25; B65D 75/26; A61B 19/026; B65D 65/38; B65D 2565/388

(57) ABSTRACT

A packaging unit for at least one pharmaceutical, medical, or cosmetic item is provided. The packaging unit includes a sterilizing chamber for accommodating the item and a prechamber that can be sealed repeatedly both from an exterior of the packaging unit and from the sterilizing chamber. The prechamber has a wall that has portions made of a wall material that is selectively permeability to one or more sterilizing agents.

### CROSS REFERENCE TO RELATED APPLICATION

This application claims benefit under 35 U.S.C. §119(a) of German Patent Application No. 10 2014 105 787.2 filed Apr. 24, 2014, the entire contents of which are incorporated herein by reference.

### BACKGROUND

1. Field of the Invention

The invention relates to a packaging unit for a pharmaceutical, medical, or cosmetic item, in particular a packaging container for at least one pharmaceutical container. The invention moreover relates to a packaging unit including the pharmaceutical, medical, or cosmetic item arranged therein, to a use of the packaging unit for sterilizing and sterile transportation of the pharmaceutical item, and to a method for sterilizing a pharmaceutical, medical, or cosmetic item that can be arranged in the packaging unit.

2. Description of Related Art

Containers such as vials, ampoules, or cartridges are widely used as a container for retaining and storing medical, pharmaceutical, or cosmetic preparations intended for administration in liquid form, in particular in pre-dosed quantities. Such containers generally have a cylindrical shape and may be made from plastics or glass. To provide for a filling process under sterile conditions as cost-efficiently as possible, the containers are packaged under sterile conditions by the manufacturer, in a packaging and transportation container. Subsequently, at a pharmaceutical company, the containers are unpacked and filled under sterile conditions, in particular in a sterile tunnel.

Often, gas-tight transportation bags are used for transporting the transportation containers. However, with the prior art transportation bags it is difficult to keep the transportation containers sterile, outside and inside, and to maintain sterility during storage or transport.

From prior art, a slider bag 100 (FIG. 1) is known, made of a plastic film and having an opening 104 that is sealable by a slider zip closure 102. A pharmaceutical item such as a drug container can be introduced into the slider bag 100 through the opening 104, and then the slider zip closure 102 can be closed. The interior of the slider bag 100 and the item disposed therein may be decontaminated via the opening 104. An adhesive write-on label 106 gives information about the item contained in the slider bag 100.

However, treatment of the exterior and interior of the slider bag 100 with different sterilizing agents is rather cost and time consuming. Furthermore, once the interior has been sterilized, the interior might be recontaminated with microorganisms upon actuation of the slider zip closure 102 for sealing the opening 104. A similar risk of contamination exists when using a vacuumizer to evacuate the slider bag 100. Moreover, when using the vacuumizer and associated film welding, the slider bag 100 cannot be reused.

### SUMMARY

The invention is based on the object to provide a packaging unit for a pharmaceutical, medical, or cosmetic item, which overcomes the aforementioned disadvantages of the prior art. More particularly, a packaging unit is to be provided, which permits to achieve and maintain high sterility of the item in an easy and efficient way.

In the present document, the OR conjunction is to be understood as a non-exclusive disjunction. Accordingly, the conjunction “A or B” is true if at least one of the involved information A, B is true.

One aspect of the invention relates to a packaging unit for a pharmaceutical item. The packaging unit may be a packaging and transportation container in which the pharmaceutical item can be packaged, stored, or transported, preferably under sterile conditions. The pharmaceutical item may be i) a pharmaceutical or medical container, or ii) a packaging container, container nest, or a tub for at least one container. The container may preferably be selected from a group comprising a drug container, a vial, an ampoule, a cartridge, a vartridge, a dual chamber cartridge, a syringe, and a dual chamber syringe.

The packaging unit may comprise a sterilizing chamber for accommodating the item and a prechamber, thus it may have a dual chamber configuration. The prechamber may be sealable both from the outside of the packaging unit and from the sterilizing chamber. That means, the prechamber may have openings towards the outside and towards the sterilizing chamber, each of which can be sealed, preferably by a re-openable closure, or by welding.

The item may be introduced into the sterilizing chamber through the openings, and once the item has been introduced the closure to the outside may be sealed in order to prevent contamination of the interior with microorganisms and to allow sterilization of the interior to start.

A wall of the prechamber may have portions comprising a wall material that exhibits selective permeability for sterilizing agents. Here, selective permeability means that the wall material is permeable for a sterilizing agent, in particular in gaseous form. Generally, textile fabrics are preferred for the wall material, the term ‘textile fabrics’ referring to woven, knitted, and especially also to nonwoven fabrics.

In the present document, ‘sterilizing’ generally refers to sterilization, disinfection, or decontamination. Preferably, when the packaging unit is subjected to a flow of a first sterilizing agent, the first sterilizing agent can penetrate through the wall material into an interior of the packaging unit to sterilize the interior. Simultaneously, the wall material can be sterilized by a second sterilizing agent, e.g. using hydrogen peroxide technology, by subjecting the packaging unit to a flow of the second sterilizing agent.

The sterilization should achieve a germ reduction of at least four orders of magnitude (or six orders of magnitude for specific applications) of a specific test microorganism suitable for the sterilization process. For example, when using the hydrogen peroxide technology, Bacillus subtilis SA22 should be used as a test microorganism.

The second sterilizing agent may be more potent and hence more toxic to living organisms than the first sterilizing agent. In this way, sterilization of the exterior is achieved faster and more effectively than that of the interior. Also, a sterilizing agent can be used for the outer surface, which does not contaminate the containers inside.

The wall material exhibiting selective permeability advantageously allows to strictly preserve sterility in the interior of the packaging unit or in the sterilizing chamber: once the item has been introduced into the packaging unit and the prechamber opening to the exterior has been sealed, microorganisms cannot get into the packaging unit any longer. Therefore, after sterilization of the interior using the first sterilizing agent, the interior or the item will remain sterile. The risk of subsequent contamination of the interior with microorganisms is zero as long as the prechamber opening remains sealed to the outside.

The dual chamber configuration of the packaging unit advantageously allows to evacuate the sterilizing chamber, in particular to a pressure of less than 200 mbar, once the prechamber opening to the sterilizing chamber has been closed. The negative pressure is a further significant improvement in sterility within the sterilizing chamber. Improvement in sterility here means a further reduction of the number of microorganisms. Thus, the present inventive packaging unit permits a two-stage reduction of the number of microorganisms: i) by sterilization, whereby microorganisms are largely destroyed; and ii) by evacuation, whereby the majority of the microorganisms remaining in the gas are pumped out. Moreover, through the evacuating the tightness of the bag is indicated more easily.

Another aspect of the invention relates to the aforementioned packaging unit including a pharmaceutical item in form of a container or in form of a packaging container or container nest or tub for at least one container. The container is preferably at least one selected from a group comprising a drug container, a vial, an ampoule, a cartridge, a vartridge, a dual chamber cartridge, a syringe, and a dual chamber syringe.

Another aspect of the invention relates to a use of the packaging unit described above for sterilizing and sterile transportation of a pharmaceutical item in form of i) a container, or ii) a packaging container, or container nest, or tub for at least one container. The container is preferably at least one selected from a group comprising a drug container, a vial, an ampoule, a cartridge, a vartridge, a dual chamber cartridge, a syringe, and a dual chamber syringe.

One aspect of the invention relates to a method for sterilizing a pharmaceutical item that can be arranged in a packaging unit. The packaging unit may comprise a sterilizing chamber and a prechamber in fluid communication with the sterilizing chamber, wherein the prechamber opens to and can be sealed from the exterior of the packaging unit and has a wall that includes portions comprising a wall material which exhibits selective permeability for sterilizing agents.

The method may comprise the steps of: i) introducing the item into the sterilizing chamber, ii) sealing the prechamber from the exterior, and iii) introducing a first sterilizing agent for which the wall material is permeable, from the exterior through the wall material into an interior of the packaging unit for sterilizing the interior or the item.

According to one embodiment, the wall material exhibiting selective permeability may be permeable for a first, in particular a gaseous sterilizing agent, and the first sterilizing agent is preferably introduceable through the wall material into an interior of the packaging unit for sterilizing the interior.

Preferably, the wall material exhibiting selective permeability may be impermeable to microorganisms.

The first sterilizing agent may comprise ethylene oxide referred to as ETO. The second sterilizing agent may comprise vaporized hydrogen peroxide referred to as VHP.

The wall material exhibiting selective permeability may comprise a textile fabric, such as in particular a nonwoven fabric which is preferably made of or includes synthetic fibers. More particularly, textile fabrics made of or including hydrophobic synthetic fibers are suitable. Plastics suitable for the selectively permeable wall material in particular include polyethylene, polypropylene, and PET.

A very suitable wall material is a polyethylene nonwoven fabric. The nonwoven fabric Tyvek® can be mentioned as an example thereof. Tyvek® is a registered trademark of DuPont for a nonwoven fabric of high density polyethylene (HDPE) that consists of fibrillated finest filaments of a diameter ranging from 0.5 to 10 μm and closely crosslinked to form networks. Based on Tyvek®, all common sterilization methods may be used, including ETO, gamma and electron beams, steam (under controlled conditions), and hydrogen peroxide plasma sterilization. Tyvek® allows sterilizing gases and steam to quickly penetrate and escape. Regardless of the sterilization method, Tyvek® provides protection as a microbial barrier and retains its strength.

The prechamber may have: a first, in particular gas-tight resealable closure for sealing the opening to the exterior, preferably in a manner so that it can be reopened; and a second, in particular gas-tight resealable closure for sealing the opening to the sterilizing chamber, preferably in a manner so that it can be reopened.

The first or second closure may be actuable from the outside to seal or open the respective openings. This advantageously permits to prevent contamination of the interior with microorganisms when actuating the closures.

The first or second closure may be provided in form of a pressure-tight closure, zip closure, or Ziploc®, preferably as a single closure or a multiple closure, Ziploc being a registered trademark originally developed and marketed by Dow Chemical for a reusable, resealable zip closure for storage bags and containers.

The packaging unit may be provided in form of a hollow thin-walled easily deformable item, preferably as a bag. A wall of the packaging unit may be resistant to VHP or ETO, and may preferably comprise a chemical-resistant plastic material, so that the bag is reusable. The plastic material may comprise a polyamide referred to as PA, or a polyethylene referred to as PE, or a polycarbonate referred to as PC, or a polypropylene referred to as PP, or a polysulfone referred to as PSU, or a polyvinyl chloride referred to as PVC.

The wall of the packaging unit may comprise a metallic foil, preferably of aluminum, or may comprise an aluminum-polypropylene composite material, which allows to achieve improved rigidity and chemical resistance.

The packaging unit, in particular the sterilizing chamber, may be adapted to be evacuated, so that preferably a gas can be pumped off from the interior of the packaging unit or sterilizing chamber to reduce a pressure inside the packaging unit or sterilizing chamber. For this purpose, the sterilizing chamber may be equipped with a vacuum port to which a vacuum pump can be connected.

According to one embodiment, at least one physical parameter in the interior of the packaging unit can be measurable or measured, in particular a parameter selected from a group comprising temperature, gas composition, humidity, pressure, and electromagnetic radiation, preferably by means of at least one sensor arranged in the packaging unit. Electromagnetic radiation preferably refers to ultraviolet radiation or gamma radiation. For better measuring UV radiation, a UV transparent window may preferably be used.

The packaging unit may comprise at least one means for altering the physical parameter, such as heating or cooling elements, or may be connectable to such means. For example, the sterilizing chamber may be connectable to an external vacuum pump, via a vacuum port. Furthermore, the packaging unit may be introducible into a gas temperature control cabinet in which appropriate conditions of temperature, pressure, gas composition, and humidity can be established. If autonomy of the packaging unit is desired, the latter may include a control means which can be used to control the physical parameter.

The packaging unit may comprise means for wireless transfer of information, preferably optical or radio link transfer. The information may relate to the physical parameter or to an identity of the pharmaceutical item. The identity may relate to one of a group comprising product name, manufacturer, quantity, date of manufacturing, processing sequence, and expiration date, and the like. The information may be conveyed to an operator or to a receiver outside the packaging unit.

The means for wireless transfer of information may i) be based on RFID or RuBee technology, or ii) comprise an electronic display or an adhesive write-on label. In the case of ii), the information may be displayed on the display or may be written on the adhesive label by a person. In the case of i), the information may be transferred to a receiver, for controlling or adjusting the physical parameter or for informing a person.

Preferably, the outer surface of the packaging unit may be subjected to a flow of a second sterilizing agent to which the wall material is impermeable, for sterilizing the outer surface. The flow around the outer surface may comprise: vaporized hydrogen peroxide which is preferably generated by evaporation from an aqueous hydrogen peroxide solution which in particular has a concentration of more than 5% and less than 50%; or a mixture of vaporized hydrogen peroxide and air, which mixture preferably includes more than 5% and less than 50% of hydrogen peroxide.

The sterilizing chamber may in particular be sealed from the outside i) by means of a closure for sealing an opening between the sterilizing chamber and the prechamber, or ii) by sealing a wall portion that comprises the wall material exhibiting selective permeability for sterilizing agents, preferably by a metallic adhesive label, such as of aluminum. The sealing using a metallic adhesive label may be implemented as an alternative to the sealing by means of the second closure.

The prechamber may be sealed from the sterilizing chamber or from the outside by welding, preferably thermal welding, hot welding, high-frequency welding, or microwave welding of the respective openings. In this case, portions of the bag film may be welded to one another.

The invention will now be described in more detail by way of exemplary embodiments and with reference to the drawings, wherein the same reference numerals designate the same or equivalent elements. The features of different exemplary embodiments may be combined.

### BRIEF DESCRIPTION OF THE FIGURES

FIG. 1 shows a prior art packaging unit;

FIGS. 2a-d illustrate operating phases for a packaging unit of the invention according to a first embodiment;

FIG. 3 illustrates a packaging unit of the invention according to a second embodiment; and

FIGS. 4a, 4b illustrate packaging units of the invention according to a third embodiment, including different pharmaceutical items.

### DETAILED DESCRIPTION OF THE INVENTION

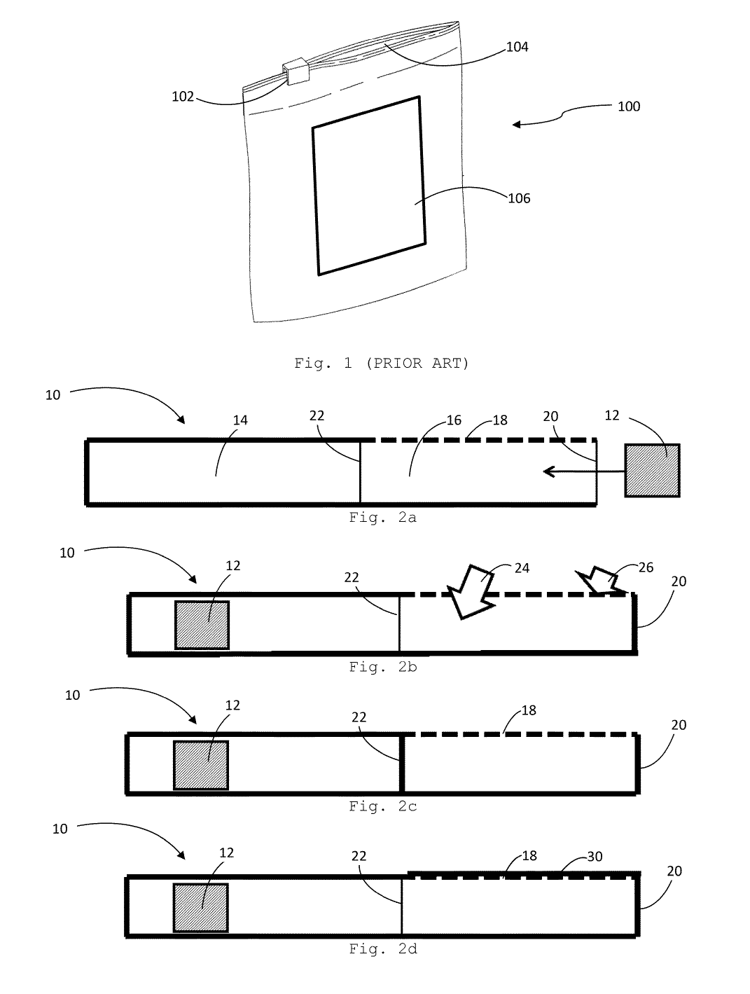

The object of the invention is to modify a prior art packaging unit 100 (see FIG. 1) so that it provides for a high level of sterility of the packaged item and at the same time is easy to use. With the inventive idea to use a multi-chamber configuration 14, 16 in which one of the chambers has a wall including portions that comprise a wall material 18 which exhibits selective permeability for sterilizing agents 24, 26, the object of the invention is achieved in an elegant way by interpreting sterilization as a reduction of the number of microorganisms by preferably at least four orders of magnitude. The packaging unit 10 according to the invention enables a two-step reduction of the number of microorganisms: i) by sterilization, whereby microorganisms are largely destroyed, and ii) by evacuation, whereby the majority of the remaining microorganisms are pumped out with the gas. Through the evacuating, tightness of the bag is indicated more easily.

The implementation of sterilization using the packaging unit 10 will be explained below with reference to FIGS. 2a-2d. These figures schematically illustrate the configuration and functionality of packaging unit 10 but do not represent naturalistic drawings.

FIG. 2a shows the packaging unit 10 of the invention for a pharmaceutical item 12. Packaging unit 10 comprises i) a sterilizing chamber 14 for accommodating the item 12, and ii) a prechamber 16 that opens to and can be sealed from an outside of the packaging unit 10 as well as to and from sterilizing chamber 14. A wall of prechamber 16 has portions comprising a wall material 18 that exhibits selective permeability for sterilizing agents 24, 26.

The wall material 18 exhibiting selective permeability for sterilizing agents 24, 26 is a textile fabric, preferably a nonwoven fabric made of synthetic fibers, more preferably a nonwoven fabric of polyethylene fibers. Tyvek® may be mentioned as an example of a suitable nonwoven fabric. Packaging unit 10 is provided in form of a plastic film bag, for example a PVC bag, which has first and second closures 20, 22 provided in form of Ziploc® closures. Closures 20, 22 are open, so that the pharmaceutical item 12 can be inserted into sterilizing chamber 14. The first sterilizing agent 24 comprises ETO, the second sterilizing agent 26 comprises VHP.

In FIG. 2a, in which all elements of packaging unit 10 can be seen, the pharmaceutical item 12 is outside the packaging unit 10. This illustrates a condition prior to sterilization.

FIG. 2b shows the packaging unit 10 into which the pharmaceutical item 12 has already been introduced; the pharmaceutical item is disposed in sterilizing chamber 14. The first closure 20 has already been sealed gas-tightly, the second closure 22 is open. Sterilization is accomplished by introducing the first sterilizing agent 24 for which the wall material 18 is permeable, through the wall material 18 into an interior of the packaging unit 10 for sterilizing the interior and the item 12. Subsequently, the outer surface may be sterilized by being subjected to a flow of the second sterilizing agent 26. Preferably, the first chamber may be closed for this purpose.

As an alternative to the sterilization of the outer surface using the first sterilizing agent 24, the outer surface may be sterilized using UVC light, gamma rays, or by hot air sterilization.

The described sequence (first sterilizing agent 24 and then second sterilizing agent 26) may be reversed by first applying the second sterilizing agent 26 and thereafter the first sterilizing agent 24. It is also possible for the packaging unit 10 to be simultaneously subjected to a flow of the two sterilizing agents, because in each case the first sterilizing agent 24 will penetrate into the packaging unit 10 and bacteria and microorganisms will be blocked.

In FIG. 2c, the second closure 22 is sealed, the first closure 20 is also sealed, though the state of the first closure 20 (open or closed) is not relevant at this stage, since the first closure 20 does not contribute to the sealing of the sterilizing chamber 14 towards the exterior, tightness thereof is produced by sealing the second closure 22. In this configuration, evacuation of the sterilizing chamber 14 to a pressure of less than 200 mbar is accomplished by pumping off through a vacuum port, not shown here, of the sterilizing chamber 14. Once evacuation is completed, the sterilization of the pharmaceutical item 12 and of the sterilizing chamber 14 is completed.

FIG. 2d shows an alternative way of establishing tightness of the sterilizing chamber 14 to the exterior: the portion including wall material 18 that exhibits selective permeability is air-tightly closed by an adhesive sealing label 30. Evacuation of the sterilizing chamber 14 may also be performed in this configuration.

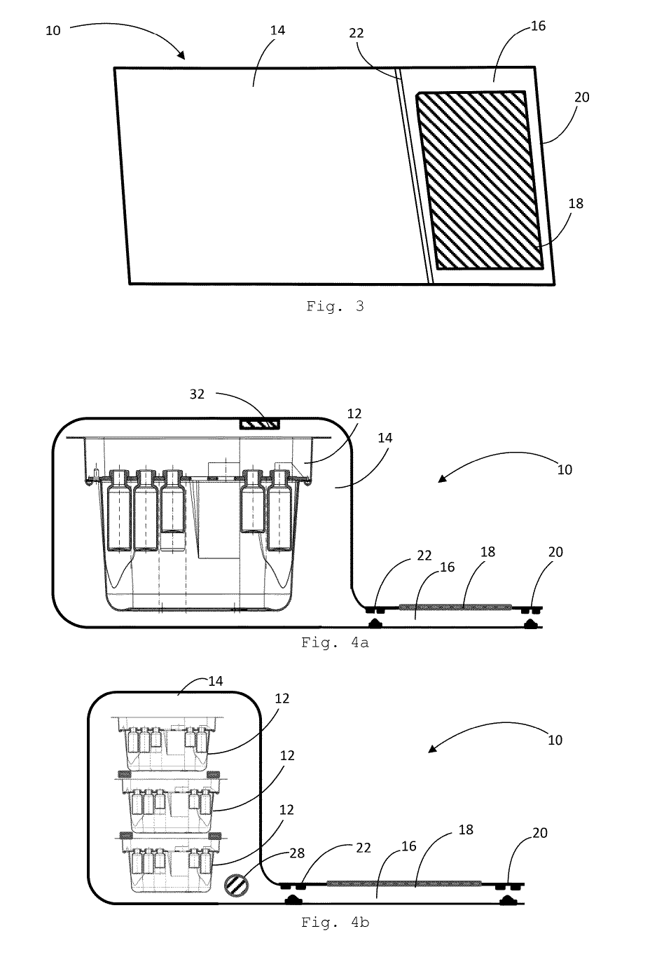

FIG. 3 is a naturalistic drawing of packaging unit 10 in a top plan view without any pharmaceutical item in the interior of the packaging unit 10. Here, it can be seen that the packaging unit 10 is provided in form of a bag.

FIGS. 4a, 4b are respective side views of packaging unit 10, with one or more pharmaceutical items 12 disposed in the sterilizing chamber 14. In FIG. 4a, the pharmaceutical item 12 is a container nest or tub for a plurality of containers in form of drug containers. In FIG. 4b, the pharmaceutical item 12 consists of a plurality of stacked container nests. In FIG. 4a a sensor 32 can be seen which can be used to measure at least one physical parameter in the interior of the packaging unit, in particular a parameter selected from a group comprising temperature, gas composition, humidity, electromagnetic radiation, and pressure. Sensor data is transferred to the outside by means of an RFID or RuBee chip 32.

The invention provides a gas-tight resealable bag 10 for transportation containers 12 for vials, syringes, dual chamber syringes, cartridges, dual chamber cartridges, and vartridges, which containers 12 are flexible enough to safely transport different sizes (length and diameter) and allow to realize a sterile pharmaceutical packaging, transportation container, and bag. The packaging can be used for cosmetic, medical, or pharmaceutical applications. The bag 10 can be completely decontaminated with respect to the outer surface, the inner surface, the interior of the container, and the contents.

The invention provides a resealable gas-tight bag 10 for transporting (during and outside of the manufacturing process) transportation containers 12 which contain pharmaceutical containers, which bag can be disinfected interiorly using ETO and exteriorly using VHP without contaminating the containers with VHP. The bag can be evacuated and is equipped with sensors which control the vacuum and other parameters, such as temperature. The present invention relates to internal and external decontamination of a packaged item per se known, in particular in the medical and pharmaceutical field.

The bag 10 provides for safe and sterile transport of transportation containers 12 for vials, syringes and dual chamber syringes, cartridges and dual chamber cartridges, and vartridges of different sizes (length and diameter). The bag 10 may be used for cosmetic, medical, or pharmaceutical applications.

The bag 10 may be repeatedly sealed using a pressure-tight closure 20, 22 or zip closure. The bag 10 can be air-tightly sealed repeatedly and enables to keep the containers sterile. The packaging may be provided with a Tyvek film 18 to enable sterilization within the package. The bag 10 may be sealed behind the pressure-tight closure/zip closure 20, 22 by thermal welding, hot welding, high-frequency welding, and/or microwave welding.

The bag 10 and/or the packaging may be provided with a fluorescent counterfeit protection, or with an RFID/RuBee chip including a sensor (e.g. O₂, humidity, temperature). The bag 10 may be provided with a sterile outer surface.

The present invention generally relates to a sterile packaging for a plurality of containers intended for storing substances for medical, pharmaceutical, or cosmetic applications, in particular vials, ampoules, or cartridges, and more particularly relates to a packaging configuration comprising a bag which permits to easily sterilize and then safely transport the containers.

According to one method for sterile packaging of containers using a bag 10 for medical, pharmaceutical, or cosmetic applications, the following steps may be performed: providing a carrier 12, arranging the plurality of containers in receptacles of the carrier 12; providing a bag 10 made of a protective film which has at least one gas-impermeable portion for packaging the receptacles in sterile manner; sealing the opening of the protective film with a zip closure 20 and, if necessary, using a clamp to provide the packaging configuration as described above.

According to one embodiment, the receptacles may subsequently be sterilized or disinfected by introducing an ETO gas flow through at least one gas-permeable portion (Tyvek®) 18 of the bag 10.

According to one embodiment, the bag 10 is made of a material which is resistant to vaporized hydrogen peroxide (VHP) (H₂O₂). Suitable for this purpose are special chemical-resistant plastics, such as polyamide (PA), polyethylene (PE), polycarbonate (PC), polypropylene (PP), PVC, and PSU. A metallic foil or aluminum foil is likewise suitable. Particularly suitable is a film comprising an aluminum-polypropylene composite material.

Hydrogen peroxide (VHP) has a sterilizing effect, can advantageously be prepared easily and at low cost by active evaporation of an aqueous hydrogen peroxide solution and may thus be used to sterilize the outer surface of the bag 10. In order to achieve a high level of biological decontamination of microorganisms, a defined high concentration of more than 5% up to 50% is required. Sterilization may also be accomplished using UVC light or gamma rays. The Tyvek® portion 18 with the remaining bag portion has to be sealed by a second zip closure 22 and/or a clamp and/or by thermal welding. Hydrogen peroxide generally has a cytotoxic and disinfecting effect, due to its high toxicity to many prokaryotic microorganisms. Following VHP decontamination, the hydrogen peroxide concentration in the containers disposed in a transportation container inside the bag is preferably not more than 0.03 ppm.

According to one method for treating or processing containers for medical, pharmaceutical, or cosmetic applications using a bag 10 and a packaging configuration as described above, the following steps may be performed: providing and opening a bag 10; introducing at least one transportation container 12 (more are also possible) that includes a nest with a plurality of receptacles each one for receiving a respective container; sealing the bag 10, preferably in sterile manner; subjecting the interior of the bag 10 to ETO gas via a gas-permeable portion (Tyvek®) 18 which however has a barrier threshold defined so as to block contaminants from passing into the interior of the enclosure; and sealing the bag behind the Tyvek portion 18.

Alternatively, the Tyvek window 18 may be sealed using an aluminum sticker 30, and/or may be sealed by a clamp, and/or by thermal welding or hot welding or high-frequency welding or microwave welding. Optionally, the bag is evacuated (<200 mbar); the outer surface of the bag is decontaminated using VHP; optionally, the bag is packaged using a second gas-tight bag.

Inside the bag or inside the transportation container within the bag, at least one electronic wireless readable RFID chip or RuBee chip may be arranged (a RuBee chip transmits on frequencies that pass through metal and water), which is adapted for non-contact readout through the side walls of the packaging unit and is responsive to a query to output information relating to the identity, important product properties (manufacturer, content, manufacturing date, expiry date, etc.), and the like. The chip may be adhered or welded to the packaging unit 10 at a suitable place inside.

According to one embodiment, the RuBee or RFID chip may be integrated in combination with further sensors adapted to monitor parameters of the transportation and packaging container relating to quality or authenticity properties of the containers stored in the transportation and packaging container over time. These quality or authenticity properties may be acquired periodically and stored in a memory associated with the chip or sensor. Electrical power supply of these electronic devices may be implemented using a grid-independent power supply provided in the transportation and packaging container, especially a battery of small dimensions, or inductively via a small conductor loop. Sensors contemplated in conjunction with the present application in particular include:
a moisture sensor with or without data logging, which periodically measures and, if desired, records the humidity prevailing in the transportation and packaging container;
a gas sensor with or without data logging, which measures and, if desired, records the concentration of gases such as O₂, ozone, CO₂, or of sterilizing gases such as ethylene oxide, formaldehyde present in the transportation and packaging container;
a temperature sensor with or without data logging, which periodically measures and, if desired, records the temperature prevailing in the transportation and packaging container;
a UV sensor with or without data logging, which periodically measures and, if desired, records the UV radiation penetrating into the transportation and packaging container;
a gamma-ray or electron beam or X-ray sensor with or without data logging, which periodically measures and, if desired, records the radiation penetrating into the transportation and packaging container;
a pressure sensor with or without data logging, which measures and, if desired, records the pressure of the gas present in the transportation and packaging container.

The gas or pressure sensors may for example be used to proof the integrity of the packaging unit and of the containers received therein.

The invention thus provides a resealable gas-tight bag 10 for transporting transportation containers 12 which accommodate pharmaceutical containers, which bag can be disinfected interiorly using ETO and exteriorly using VHP without contaminating the containers with VHP. The bag 10 is evacuable and is equipped with sensors 32 that measure the vacuum and, e.g., temperature.

FIG. 3 shows a bag 10 having two zip closures 20, 22 and a gas-permeable portion 18 (Tyvek® film). The bag 10 has at least one gas-permeable portion 18 which is resealable gas-tightly on both sides thereof. For this purpose, a pressure-tight closure or zip closure may be used. Via the Tyvek® portion, the contents of the bag may be sterilized using gas (e.g. ETO). The resealable bag may be made, for example, of polyethylene LD (LDPE), aluminum, or polypropylene (PP). MDPE, HDPE, COEX, composite films including recycled material are likewise possible. Coloring may offer advantages (solarization, marking, batch encoding). The coloring may be opaque.

FIG. 4a shows a sectional view of a bag 10 containing a transportation container 12 (nest, tub, and pharmaceutical containers/vials), the bag comprising a sensor and an RFID or RuBee chip 32, two zip closures 20, 22, and a gas-permeable portion 18 (Tyvek® film). A first pressure-tight closure 20 is sealed so that an influx of gas is only possible through the gas-permeable Tyvek® portion. The second pressure-tight closure 22 may be designed so that the lower region is bent so that gas influx is facilitated.

FIG. 4b shows a sectional view of a bag containing three transportation containers 12 (nest, tub, and pharmaceutical containers/vials), the bag comprising two zip closures 20, 22 and a gas-permeable portion 18 (Tyvek® film). The pressure-tight closures have not yet been sealed. The bag 10 has a sealable vacuum port 28 through which the bag 10 can be evacuated.

### REFERENCE NUMERALS

100 Slider bag, prior art packaging unit
102 Slider zip closure
104 Opening
106 Adhesive label
10 Packaging unit, bag
12 Pharmaceutical item
14 Sterilizing chamber
16 Prechamber
18 Wall material with selective permeability for sterilizing agents
20 First closure
22 Second closure
24 First sterilizing agent
26 Second sterilizing agent
28 Vacuum port
30 Adhesive sealing label
32 Sensor with RFID/RuBee

What is claimed is:

1. A packaging unit for at least one pharmaceutical, medical, or cosmetic item, comprising:
a sterilizing chamber for accommodating the item; and
a prechamber that is sealable both from an exterior of the packaging unit and from the sterilizing chamber,
wherein the prechamber has a wall that has portions comprising a wall material exhibiting selective permeability for sterilizing agents to sterilize of an interior of the packaging unit and/or the item.

2. The packaging unit as claimed in claim 1, wherein the wall material exhibits selective permeability to a gaseous sterilizing agent or to a microorganism.

3. The packaging unit as claimed in claim 1, wherein the sterilizing agent comprises gaseous ethylene oxide.

4. The packaging unit as claimed in claim 1, wherein the wall material comprises a nonwoven fabric.

5. The packaging unit as claimed in claim 1, wherein the prechamber comprises a first gas-tight closure for sealing an opening to the exterior or a second a gas-tight closure for sealing an opening to the sterilizing chamber.

6. The packaging unit as claimed in claim 5, wherein the first or second gas-tight closure is a pressure-tight closure or a zip closure.

7. The packaging unit as claimed in claim 1, wherein the packaging unit is a device selected from the group consisting of a bag, a container, a packaging container, a container nest, and tub for at least one container.

8. The packaging unit as claimed in claim 1, wherein the wall that is resistant to VHP or ETO.

9. The packaging unit as claimed in claim 1, wherein the wall comprises a chemical-resistant plastic material selected from the group consisting of polyamide, polyethylene, polycarbonate, polypropylene, polysulfone, and polyvinyl chloride.

10. The packaging unit as claimed in claim 1, wherein the wall comprises a metallic foil or of an aluminum-polypropylene composite material.

11. The packaging unit as claimed in claim 1, wherein the sterilizing chamber is evacuable.

12. The packaging unit as claimed in claim 1, further comprising at least one sensor configured to measure at least one physical parameter of the interior, the parameter being selected from the group consisting of temperature, gas composition, humidity, electromagnetic radiation, and pressure.

13. The packaging unit as claimed in claim 1, further comprising a device to alter or control or transmit at least one physical parameter of the interior, the parameter being selected from the group consisting of temperature, gas composition, humidity, electromagnetic radiation, and pressure.

14. A method for sterilizing a pharmaceutical item that can be arranged in a packaging unit, the method comprising the steps of:
introducing the item into a sterilizing chamber of the packaging unit;
sealing a prechamber of the packaging unit from an exterior; and
introducing a first sterilizing agent, for which a wall material of a portion of the prechamber is permeable, from the exterior through the wall material into an interior of the packaging unit to sterilize the interior and/or the item.

15. The method as claimed in claim 14, further comprising subjecting an outer surface to a flow of a second sterilizing agent to which the wall material is impermeable to sterilize the outer surface.

16. The method as claimed in claim 14, further comprising sealing the sterilizing chamber from the outside by a closure sufficient to seal an opening between the sterilizing chamber and the prechamber.

17. The method as claimed in claim 14, further comprising sealing the sterilizing chamber from the outside by sealing a wall portion that comprises the wall material with a metallic adhesive label.

18. The method as claimed in claim 14, further comprising sealing the prechamber from the sterilizing chamber or from the outside by welding.

19. The method as claimed in claim 14, further comprising evacuating the interior to a pressure of less than 200 mbar.

20. The method as claimed in claim 14, further comprising measuring and/or controlling at least one physical parameter in the interior, the parameter being selected from the group consisting of temperature, pressure, gas composition, electromagnetic radiation, and humidity.
